(19) United States
(12) Patent Application Publication
(10) Pub. No.: US 2003/0206610 A1
(43) Pub. Date: Nov. 6, 2003

(54) Patient positioning system

(76) Inventor: William F. Collins, Clayton, CA (US)

Correspondence Address: Siemens Corporation, Attn: Elsa Keller, Legal Administrator, Intellectual Property Department, 186 Wood Avenue South, Iselin, NJ, 08830, US

(21) Appl. No.: 10/252,912

(22) Filed: Sep. 23, 2002

Related U.S. Application Data
(60) Provisional application No. 60/377,352, filed on May 1, 2002.

(51) Int. Cl. A61N 5/10

(52) U.S. Cl. 378/65

(57) ABSTRACT

A system includes a radiation source for emitting radiation, an imaging device for generating three-dimensional data representing internal portions of a patient in conjunction with the emitted radiation, and a processor for determining a correspondence between the generated three-dimensional data and other data representing the internal portions of the patient, and for determining whether the treatment equipment is properly positioned relative to the patient based on the correspondence.

### CROSS-REFERENCE TO RELATED APPLICATIONS

This application claims priority to U.S. Provisional Application No. 60/377,352, filed May 1, 2002 and entitled “System and Method of Focused Orthovoltage Technology for Radiotherapy”.

### BACKGROUND

1. Field

The present invention relates generally to radiation treatment, and more particularly to facilitating patient positioning during such treatment.

2. Description

Conventional radiation treatment typically involves directing a radiation beam at a tumor located within a patient. The radiation beam is intended to deliver a predetermined dose of treatment radiation to the tumor according to an established treatment plan. The goal of such treatment is to kill tumor cells through ionizations caused by the radiation.

Healthy tissue and organs are often in the treatment path of the radiation beam during radiation treatment. The healthy tissue and organs must be taken into account when delivering a dose of radiation to the tumor, thereby complicating determination of the treatment plan. Specifically, the plan must strike a balance between the need to minimize damage to healthy tissue and organs and the need to ensure that the tumor receives an adequately high dose of radiation. In this regard, cure rates for many tumors are a sensitive function of the radiation dose they receive.

Treatment plans are therefore designed to maximize radiation delivered to a target while minimizing radiation delivered to healthy tissue. However, a treatment plan is designed assuming that relevant portions of a patient will be in a particular position relative to a treatment device during treatment. If the relevant portions are not positioned exactly as required by the treatment plan, the goals of maximizing target radiation and minimizing healthy tissue radiation may not be achieved. More specifically, errors in positioning the patient can cause the delivery of low radiation doses to tumors and high radiation doses to sensitive healthy tissue. The potential for misdelivery increases with increased positioning errors.

Due to the foregoing, treatment plans are designed under the assumption that positioning errors may occur that may result in misdelivery of radiation. Treatment plans compensate for this potential misdelivery by specifying lower doses or smaller beam shapes (e.g., beams that do not radiate edges of a tumor) than would be specified if misdelivery was not expected. Such compensation may decrease as margins of error in patient positioning decrease.

It would therefore be beneficial to provide a system and method that increases the accuracy of patient positioning during radiation treatment. When used in conjunction with conventionally-designed treatments, more accurate positioning may reduce chances of harming healthy tissue. More accurate patient positioning may also allow the use of more aggressive treatments. Specifically, if a margin of error in patient positioning is known to be small, treatment may be designed to safely radiate a greater portion of a tumor with higher doses than in scenarios where the margin of error is larger.

### SUMMARY

To address at least the above problems, some embodiments of the present invention provide a system, method, apparatus, and means to generate three-dimensional data representing internal portions of a patient using treatment equipment, determine a correspondence between the generated three-dimensional data and other data representing the internal portions of the patient, and determine whether the treatment equipment is properly positioned relative to the patient based on the correspondence. Three-dimensional data may be generated from image data generated at a plurality of positions of a treatment head. In a further aspect, some embodiments may include generation of data representing surface features of the patient, wherein the step to determine whether the treatment equipment is properly positioned comprises a determination of a correspondence between the data representing surface features of the patient and other data representing surface features of the patient.

In additional aspects, provided is a system including a radiation source for emitting radiation, an imaging device for generating three-dimensional data representing internal portions of a patient in conjunction with the emitted radiation, and a processor for determining a correspondence between the generated three-dimensional data and other data representing the internal portions of the patient, and for determining whether the treatment equipment is properly positioned relative to the patient based on the correspondence.

The present invention is not limited to the disclosed embodiments, however, as those skilled in the art can readily adapt the teachings herein to create other embodiments and applications.

### BRIEF DESCRIPTION OF THE DRAWINGS

The exact nature of this invention, as well as its objects and advantages, will become readily apparent from consideration of the following specification as illustrated in the accompanying drawings, in which like reference numerals designate like parts, and wherein:

FIG. 1 is a diagram illustrating a radiation treatment room according to some embodiments of the present invention;

FIG. 2 is a diagram illustrating a radiation-focusing lens according to some embodiments of the present invention;

FIG. 3 comprises a flow diagram illustrating process steps according to some embodiments of the present invention; and

FIG. 4 is a view of a diagnostic computed tomography (CT) device.

### DETAILED DESCRIPTION

The following description is provided to enable any person skilled in the art to make and use the invention and sets forth the best modes contemplated by the inventor for carrying out the invention. Various modifications, however, will remain readily apparent to those in the art.

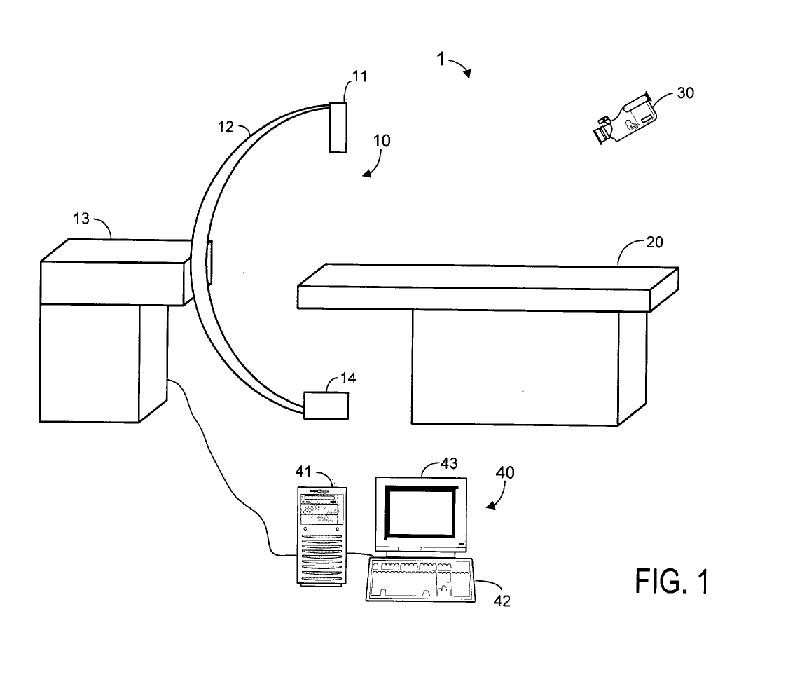

FIG. 1 illustrates radiation treatment room 1 pursuant to some embodiments of the present invention. Radiation treatment room 1 includes kilovoltage radiation treatment unit 10, treatment table 20, surface imager 30 and operator station 40. The elements of radiation treatment room 1 are used to deliver kilovoltage radiation to a patient according to a treatment plan. In this regard, kilovoltage radiation refers herein to any radiation having energies ranging from 50 to 150 keV. However, it should be noted that some embodiments of the present invention may be used in conjunction with a radiation beam of any type or intensity.

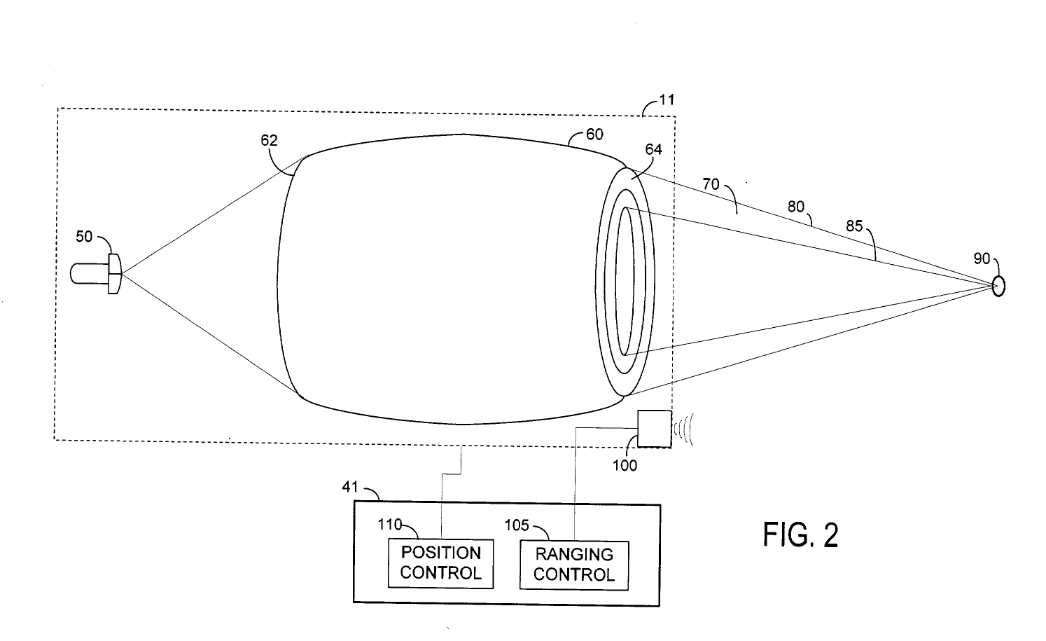

Treatment unit 10 is used to deliver treatment radiation to a treatment area and includes treatment head 11, c-arm 12, base 13 and imaging system 14. Treatment head 11 includes a beam-emitting device such as an x-ray tube for emitting kilovoltage radiation used during unit calibration and/or actual treatment. The radiation may comprise electron, photon or any other type of radiation. Treatment head 11 also includes a cylinder in which are disposed elements such as a focusing lens for optically processing the emitted radiation and ranging devices for determining a position of treatment head 11 in accordance with some embodiments of the invention. Treatment head 11 will be described in more detail below with respect to FIG. 2.

C-arm 12 is slidably mounted on base 13 and can be moved in order to move treatment head 11 with respect to table 20 and, more particularly, with respect to a patient positioned on table 20. In some embodiments, base 13 also includes a high-voltage generator for supplying power used by treatment head 11 to generate kilovoltage radiation. Many c-arm/base configurations may be used in conjunction with some embodiments of the present invention, including configurations in which base 13 is rotatably mounted to a ceiling of room 1, configurations in which one c-arm is slidably mounted on another c-arm, and configurations incorporating multiple independent c-arms.

Examples of c-arm kilovoltage radiation units include Siemens SIREMOBIL™, MULTISTAR™, BICOR™ and POLYSTAR™ units as well as other units designed to perform tomography and/or angiography. These units are often less bulky and less costly than megavoltage radiation systems. Of course, any system for delivering a focused radiation beam may be used in conjunction with some embodiments of the present invention.

Imaging system 14 acquires an image based on the radiation emitted by treatment head 11. The image reflects the attenuative properties of objects located between treatment head 11 and imaging system 14 while the radiation is emitted. The acquired image may represent internal portions of a patient and be used to confirm that the patient is positioned in accordance with a treatment plan.

Imaging system 14 may comprise an image intensifier and a camera. An image intensifier is a vacuum tube that converts X-rays to visible light, which is then detected by the camera to produce an image. Imaging system 14 may also comprise a flat-panel imaging system that uses a scintillator and solid-state amorphous silicon sensors to produce an image based on received radiation. The RID 1640, offered by PerkinElmer®, Inc. of Fremont, Calif., is one suitable device.

A patient is placed on treatment table 20 during treatment in order to position a target area located within the patient between treatment head 11 and imaging system 14. Accordingly, table 20 may comprise mechanical systems for moving itself (and thereby the patient) with respect to unit 10. In some embodiments, the patient and/or treatment head 11 are moved to several different positions relative to one another. At each position, radiation is emitted from treatment head 11 and received by imaging system 14 in order to produce a two-dimensional data set representing internal portions of the patient. Known image processing techniques may be applied to the two-dimensional data sets to generate three-dimensional data representing internal portions of the patient.

Surface imager 30 acquires data representing surface features of a patient positioned on table 20. As will be described in detail below, the acquired data may be used to determine whether the patient is properly positioned. The data may comprise range data and may be acquired using any suitable technique, such as stereo video acquisition or time-of-flight laser detection. In some embodiments, surface imager 30 acquires the data by projecting a light pattern onto a surface and by sensing how the light pattern coats the surface. Of course, data acquired by surface imager 30 need not be in a range data format; any format usable to represent surface features will suffice, including any standard video format.

Operator station 40 includes processor 41 in communication with an input device such as keyboard 42 and an output device such as operator display 43. Operator station 40 is typically operated by an operator who administers actual delivery of radiation treatment as prescribed by an oncologist. Operator station 40 may be located apart from treatment unit 10, such as in a different room, in order to protect the operator from radiation. It should be noted, however, that kilovoltage radiation treatment does not require protective measures to the extent of those taken during megavoltage radiation therapy, resulting in less costly therapy.

Processor 41 may store processor-executable process steps according to some embodiments of the present invention. In some aspects, the process steps are executed by operator station 40, treatment unit 10, imaging system 14, and/or another device to generate three-dimensional data representing internal portions of a patient using treatment equipment, determine a correspondence between the generated three-dimensional data and other data representing the internal portions of the patient, and determine whether the treatment equipment is properly positioned relative to the patient based on the correspondence.

In some aspects, the process steps may also be executed to generate data representing surface features of the patient, wherein the determination of whether the treatment equipment is properly positioned comprises a determination of a correspondence between the data representing surface features of the patient and other data representing surface features of the patient.

The above-described steps may also be embodied, in whole or in part, by hardware and/or firmware of processor 31, treatment unit 10, treatment head 11, imaging system 14, surface imager 30, and another device. Of course, each of the devices shown in FIG. 1 may include less or more elements than those shown. In addition, embodiments of the invention are not limited to the devices shown.

FIG. 2 is a representative view of elements of treatment head 11 according to some embodiments of the present invention. It should be noted that the neither the elements nor their physical relationships to one another are necessarily drawn to scale. As shown, treatment head 11 includes x-ray tube 50 for emitting radiation toward lens 60. In some embodiments, x-ray tube 50 comprises a Diabolo™ x-ray tube. The radiation enters entry surface 62 of lens 60 and some or all of the radiation exits exit surface 64. In this regard, the radiation energy exiting exit surface 64 may comprise 10% or less of the total radiation energy striking entry surface 62.

Lens 60 comprises strips of reflective material arranged in the form of one or several barrels nested around a central axis. The reflective material may comprise Highly Oriented Pyrolitic Graphite (HOPG), which consists of planes of carbon atoms that are highly oriented toward one another. In the ideal variant, these planes are parallel to one another. Each “barrel” in a multiple barrel lens is separated from adjacent “barrels” by Plexiglas or another optically neutral substrate. Lens 60 may comprise any type of lens, including but not limited to radiation-focusing lenses such as those described in U.S. Pat. No. 6,359,963 to Cash, in U.S. Pat. No. 5,604,782 to Cash, Jr., in U.S. Patent Application Publication No. 2001/0043667 of Antonell et al., and/or elsewhere in currently or hereafter-known art. In some embodiments, treatment head 11 does not include a lens.

By virtue of the composition, shape and construction of lens 60 and of properties of the radiation emitted by x-ray tube 50, radiation exiting from exit surface 64 substantially follows radiation path 70. Geometrically, path 70 comprises a hollow conical volume between outer cone surface 80 and inner cone surface 85. Of course, different lenses used in conjunction with embodiments of the invention may direct radiation along differently-shaped paths.

Lens 60 operates to substantially focus all or a portion of the directed radiation on focal area 90. Focal area 90 may comprise a point in space or a larger area. In some embodiments of lens 60, focal area 90 is approximately 1 cm in diameter. According to the FIG. 2 embodiment, focal area 90 is spaced from an exit surface of lens 60 by a distance determined by the composition, shape and construction of lens 60 as well as by characteristics of the radiation emitted by x-ray tube 50.

It should be noted that path 70 might not terminate at focal area 90. Rather, path 70 may continue thereafter, becoming further attenuated and unfocused as its distance from focal area 90 increases. In some embodiments, the divergence of path 70 from focal area 90 roughly mirrors its convergence thereto.

Range detecting device 100 generates data usable to determine a distance between an end of treatment head 11 and a patient. A distance between the end of treatment head 11 and exit surface 64 is known and a distance between an end of treatment head 11 and x-ray tube 50 is known. Accordingly, the data generated by range detecting device 100 may also be usable to determine a distance between exit surface 64 and the patient and a distance between x-ray tube 50 and the patient. Any of these distances may be used to determine whether the patient is properly positioned and/or to change a position of treatment unit 10 relative to the patient in accordance with a treatment plan.

Range detecting device may comprise a laser ranging device or another currently or hereafter-known device for generating a range data. It should be noted that range detecting device 100 may be configured differently than as shown in FIG. 2. Generally, range detecting device 100 may be arranged in any manner that allows operation in accordance with embodiments of the present invention.

Range detecting device 100 may be controlled by ranging control 105 of processor 41. In some embodiments, ranging control 105 requests data from range detecting device 100 and a position of treatment unit 10 relative to a patient is changed based thereon. Ranging control 105 may comprise one or more of software, hardware, and firmware elements to control range detecting device 100 according to some embodiments of the invention. Of course, ranging control 105 may be located in other devices, such as treatment head 11, base 13, a stand-alone device, or another device.

In this regard, position control 110 may be used to control a position of treatment unit 10 relative to a patient lying on table 20. According to some embodiments, position control 110 is used to change a position of treatment head 11 relative to the patient based on a correspondence between three-dimensional data representing internal portions of the patient generated using treatment head 11 and other data representing internal portions of the patient. Position control 110 may operate to change the position by movement of treatment head 11, c-arm 12, table 20, and/or another element of treatment unit 10. As described with respect to ranging control 105, position control 110 need not be located within processor 41.

Treatment head 11 may also include beam-shaping devices such as one or more jaws, collimators, reticles and apertures. These devices may be used to change the shape of path 70 and to thereby also change the shape and/or position of focal area 90. The devices may be placed between lens 60 and focal area 90 and/or between x-ray tube 50 and lens 60.

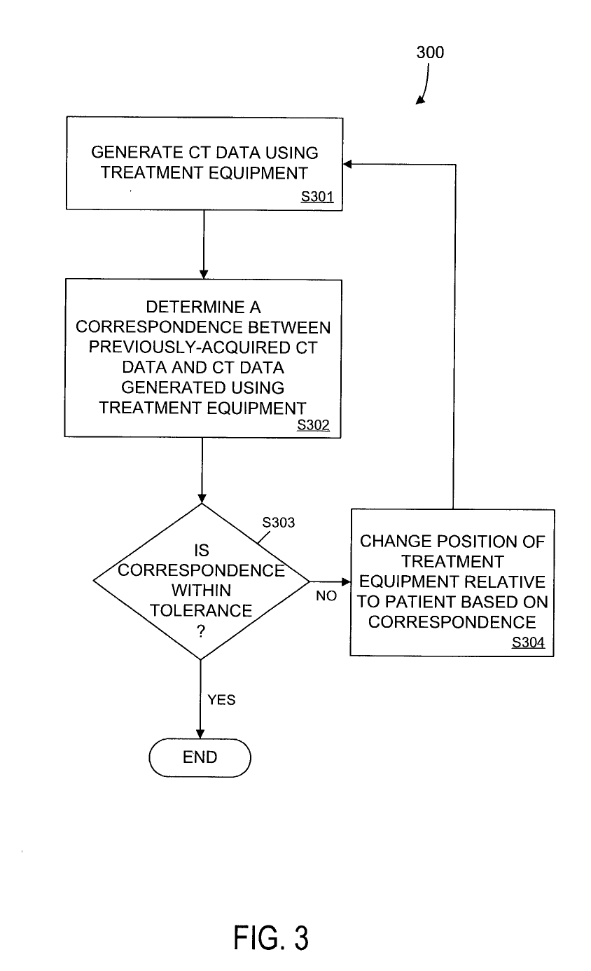

FIG. 3 comprises a flow diagram of process steps 300 according to some embodiments of the invention. Process steps 300 may be embodied in hardware, firmware, and/or software of processor 41, treatment unit 10, table 20, and/or another device.

Process steps 300 begin at step S301, in which computed tomography (CT) data is generated using treatment equipment. The CT data comprises three-dimensional data representing internal portions of a patient lying on table 20. Other types of three-dimensional data representing internal portions of the patient may also or alternatively be generated in step S301.

In some embodiments of step S301, the patient is placed between treatment head 11 and imaging system 14. Treatment head 11 emits radiation that is variously attenuated by portions of the patient lying between head 11 and system 14. The attenuated radiation is detected by imaging system 14 and used to generate a two-dimensional image of internal portions of the patient. Treatment head 11 and imaging system 14 are then rotated to different angles relative to the patient and subsequent two-dimensional images are similarly acquired. Processor 41 generates the three-dimensional data of step S301 based on the acquired two-dimensional dimensional images using currently- or hereafter-known techniques, such as cone beam reconstruction using a Feldkamp algorithm.

Also generated in step S301 may be data representing surface features of the patient. This data may be generated by surface imager 30, and may reflect surface features of the patient in a case that the patient is in a particular position, wherein the particular position is substantially identical to a position of the patient during the above-described generation of the CT data.

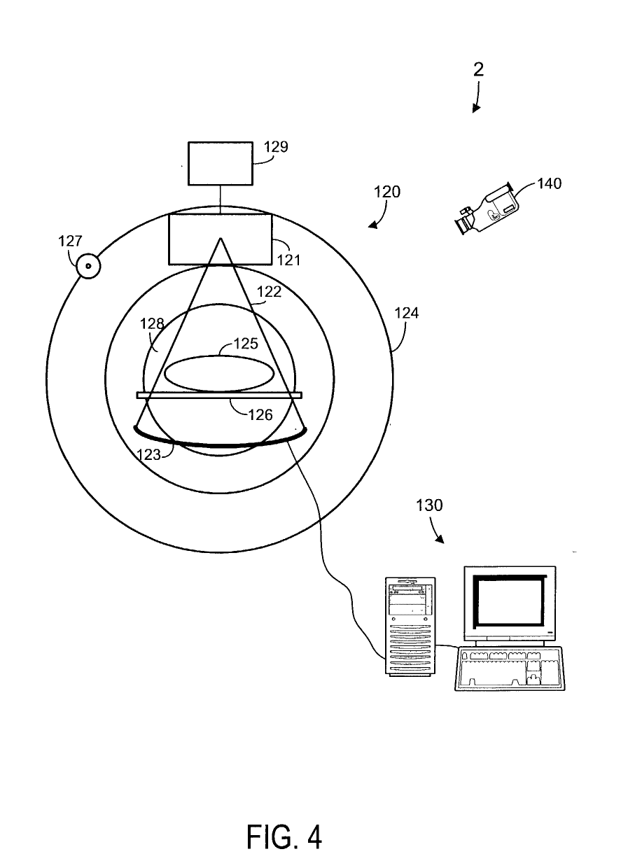

Next, in step S302, a correspondence between previously-acquired CT data and the CT data generated in step S301 is determined. The correspondence may be determined in step S302 using currently- or hereafter-known algorithms. In some embodiments, the previously-acquired CT data also represents the internal portions of the patient, but was obtained prior to step S301 using a CT scanner such as CT scanner 120 of FIG. 4.

CT scanner 120 is located in CT room 2 and may be operated prior to step S301 to obtain data for diagnosing a patient and/or for planning radiation treatment. CT scanner 120 includes x-ray source 121 for emitting fan-shaped x-ray beam 122 toward radiation receiver 123. Both x-ray source 121 and radiation receiver 123 are mounted on ring 124 such that they may be rotated through 360 degrees while maintaining the physical relationship therebetween. In order to acquire data representing structures internal to a patient (i.e., CT data), patient 125 lies on patient bed 126. Next, x-ray source 121 and receiver 123 are rotated by rotation drive 127 around a measurement field 128 in which patient 124 lies.

During this rotation, x-ray source 121 is powered by high-voltage generator 129 to transmit radiation toward receiver 123. At predetermined rotational angle positions, receiver 123 produces sets of data and the sets of data are transmitted to computer system 130. Computer system 130 calculates attenuation coefficients of predetermined image points from the registered data sets to generate data representing internal portions of patient 125.

In some embodiments of step S302, the data generated by CT scanner 120 is used to simulate and plan radiation treatment. Accordingly, it may be beneficial to ensure that relevant portions of the patient are positioned during radiation treatment substantially similarly to how those portions were positioned during generation of the data by CT scanner 120. Such CT simulation data may be generated similarly to the diagnostic data described above, but may be acquired using different acquisition parameters of CT scanner 120.

According to some embodiments, also determined in step S302 is a correspondence between data representing surface features of the patient and previously-acquired other data representing surface features of the patient. The other data may be acquired by surface imager 140 located proximate to CT scanner 120. Moreover, the other data may be acquired with the patient in a particular position, wherein the particular position is substantially the same as the patient's position during acquisition of the CT data by CT scanner 120. In some embodiments, the other data representing surface features of the patient is acquired at substantially a same time as the other data representing internal portions of the patient.

The correspondence between the data representing surface features may be determined using image matching algorithms. Such algorithms may compensate for differences between the position of surface imager 30 relative to a patient in treatment room 1 and the position of surface imager 140 relative to patient 125. The differences may include differences in distance and rotation. Accordingly, image-matching algorithms may initially apply a translation to one of the sets of data representing surface features in order to compensate for these differences.

In step S303, it is determined whether the correspondence is within prescribed tolerances. This determination may be based on a correspondence between data representing internal portions of the patient. The determination may also be based on a correspondence between data representing surface features of the patient. Each correspondence may be associated with a respective tolerance used in step S303, and/or the correspondences may be used to determine an overall correspondence that is compared against an overall tolerance in step S303.

If it is determined that the correspondence is not within prescribed tolerances, it is assumed that the patient is not positioned in accordance with a treatment plan. Accordingly, in step S304, a position of the treatment equipment relative to the patient is changed in step S304. This position change may be performed by moving the treatment equipment and/or the patient. The goal of this position change may be to establish a position of the treatment equipment relative to the patient that more closely matches a treatment plan developed based on the previously-acquired CT data.

In some embodiments of step S304, position control 110 operates to move c-arm 12 and table 20 based on the determined correspondence. Position control 110 may operate in conjunction with ranging control 105 in step S304. More specifically, ranging control 105 may determine a distance between the patient and tube 50, head 11, or another element of treatment unit 10 based on data received from range detecting device 100. This distance may be used by position control 110 to properly position treatment equipment such as elements of treatment unit 10 relative to the patient.

After step S304, flow returns to step S301 to generate new CT data reflecting the changed position using the treatment equipment. Flow therefore cycles between steps S301 and S304 until it is determined in step S303 that a correspondence between CT data generated using the treatment equipment and the previously-acquired CT data is within the specified tolerance.

Those in the art will appreciate that various adaptations and modifications of the above-described embodiments can be configured without departing from the scope and spirit of the invention. For example, the change of position in step S304 may be monitored by an operator using display 43 and/or controlled by the operator using an input device of operator station 40 such as keyboard 42. Similarly, imaging system 14 may acquire images during treatment and the images may be presented on display 43 so that an operator can verify patient position during treatment. In this regard, process steps 300 may be performed periodically during treatment to confirm patient positioning. In some embodiments, kilovoltage radiation treatment unit 10 may be first operated in a planning mode of operation to establish a patient position, and then transitioned to a treatment mode of operation to deliver a course of treatment. In some embodiments, an operator may confirm patient positioning during the treatment mode of operation by entering a verification mode in which process steps 300 are performed. Pursuant to some embodiments, the planning mode, the treatment mode, and the verification mode may be performed without need for an operator to enter treatment room 1 and mount or otherwise attach any accessories to treatment head 11, thereby allowing accurate and efficient patient positioning during planning and treatment.

Moreover, it should be noted that functions ascribed to one device herein may be performed by other devices. In one example, the functions ascribed to treatment unit 10 and to CT scanner 120 are performed by a single computing device. In other examples, elements or functions described with respect to one device are present in or performed by another. Therefore, it is to be understood that, within the scope of the appended claims, the invention may be practiced other than as specifically described herein.

What is claimed is:

1. A method, comprising:
(a) positioning a treatment head and an imaging device and directing an amount of kilovoltage radiation from the treatment head toward a patient positioned between the treatment head and the imaging device;
(b) generating image data based on attenuated radiation detected by the imaging device;
(c) repeating steps (a) and (b) to generate image data for a plurality of positions of the treatment head;
(d) generating three-dimensional data representing internal portions of the patient based on the image data;
(e) determining a correspondence between the generated three-dimensional data and other data representing the internal portions of the patient; and
(f) determining whether treatment equipment is properly positioned relative to the patient based on the correspondence.

2. A method according to claim 1, wherein steps (a) through (f) are performed in a planning mode of operation.

3. A method according to claim 1, further comprising selecting a treatment mode of operation once the treatment equipment is properly positioned relative to the patient.

4. A method according to claim 4, further comprising selecting a verification mode of operation during said treatment mode of operation and repeating steps (a) though (f) to verify whether treatment equipment is properly positioned relative to the patient.

5. A method according to claim 1, further comprising changing a position of the treatment equipment relative to the patient based on the correspondence.

6. A method according to claim 5, wherein the step of changing a position of the treatment equipment comprises determining a distance between the treatment equipment and the patient.

7. A method according to claim 1, further comprising:
generating data representing surface features of the patient,
wherein the step of determining whether the treatment equipment is properly positioned comprises determining a correspondence between the data representing surface features of the patient and other data representing surface features of the patient.

8. A method according to claim 7, wherein the other data representing surface features represents surface features of the patient with the patient in a position and the other data representing internal portions of the patient represents internal portions of the patient with the patient substantially in the position.

9. A method according to claim 7, wherein the other data representing surface features of the patient was generated at substantially a same time as the other data representing internal portions of the patient.

10. A medium storing processor-executable process steps, the process steps comprising:
a step to position a treatment head and an imaging device and direct an amount of kilovoltage radiation from the treatment head toward a patient positioned between the treatment head and the imaging device;
a step to generate image data based on attenuated radiation detected by the imaging device, the steps to position and to generate repeated to generate image data for a plurality of positions of the treatment head;
a step to generate three-dimensional data representing internal portions of the patient based on the image data;
a step to determine a correspondence between the generated three-dimensional data and other data representing the internal portions of the patient; and
a step to determine whether the treatment equipment is properly positioned relative to the patient based on the correspondence.

11. A medium according to claim 10, the process steps further comprising:
a step to generate data representing surface features of the patient,
wherein the step to determine whether the treatment equipment is properly positioned comprises a step to determine a correspondence between the data representing surface features of the patient and other data representing surface features of the patient.

12. An apparatus comprising:
a memory storing processor-executable process steps; and
a processor in communication with the memory and operative in conjunction with the stored process steps to:
position a treatment head and an imaging device and direct an amount of kilovoltage radiation from the treatment head toward a patient positioned between the treatment head and the imaging device;
generate image data based on attenuated radiation detected by the imaging device, the steps to position and to generate repeated to generate image data for a plurality of positions of the treatment head;
generate three-dimensional data representing internal portions of the patient based on the image data;
determine a correspondence between the generated three-dimensional data and other data representing the internal portions of the patient; and
determine whether the treatment equipment is properly positioned relative to the patient based on the correspondence.

13. An apparatus according to claim 12, the processor further operative in conjunction with the stored process steps to generate data representing surface features of the patient,
wherein determination of whether the treatment equipment is properly positioned comprises determination of a correspondence between the data representing surface features of the patient and other data representing surface features of the patient.

14. A system comprising:
a radiation source for emitting radiation from a plurality of positions directed toward a patient;
an imaging device for generating three-dimensional data representing internal portions of a patient in conjunction with the emitted radiation from the plurality of positions; and
a processor for determining a correspondence between the generated three-dimensional data and other data representing the internal portions of the patient, and for determining whether the treatment equipment is properly positioned relative to the patient based on the correspondence.

15. A system according to claim 14, further comprising:
at least one range detecting device for generating data usable to determine a distance between the patient and the radiation source.

16. A system according to claim 15, wherein the processor determines whether the treatment equipment is properly positioned relative to the patient based on the data usable to determine a distance between the patient and the radiation source.

17. A system according to claim 14, further comprising:
a surface imager for generating data representing surface features of the patient,
wherein the determination of whether the treatment equipment is properly positioned comprises determination of a correspondence between the data representing surface features of the patient and other data representing surface features of the patient.
